(19) United States
(12) Patent Application Publication
(10) Pub. No.: US 2013/0222764 A1
(43) Pub. Date: Aug. 29, 2013

(54) VISION TESTING SYSTEM

(71) Applicant: DigitalVision, LLC, (US)

(72) Inventors: Keith P. Thompson, Atlanta, GA (US); Jose R. Garcia, Mableton, GA (US)

(73) Assignee: DigitalVision, LLC, Atlanta, GA (US)

(21) Appl. No.: 13/778,367

(22) Filed: Feb. 27, 2013

Related U.S. Application Data
(60) Provisional application No. 61/604,310, filed on Feb. 28, 2012.

(51) Int. Cl. A61B 3/18

(52) U.S. Cl. CPC A61B 3/18
USPC 351/209; 351/211; 351/246

(57) ABSTRACT

A vision testing system comprises a image wavefront modulator, eye tracking system, focusing system using a spherical concave mirror, and a patient station. In various embodiments, the image wavefront modulator and the patient's eyes are positioned off axis with respect to the optical axis of the focusing mirror. Thus, optical elements in the wavefront modulator may automatically adjust to correct for aberrations introduced by the focusing system. Moreover, the optical elements may also be used to automatically correct for magnification errors introduced by movement of the patient within the patient testing station. Furthermore, the eye tracking system may be used to determine the errors introduced by movement of the patient eyes. Finally, the wavefront modulator may be used to produce an image on the patient's retina that accurately emulates an image that result if the patient was looking through a spectacle lens of a particular design during various gaze angles.

### CLAIM OF PRIORITY

This application claims the benefit of, and incorporates by reference in its entirety, U.S. Provisional Patent Application No. 61/604,310, filed Feb. 28, 2012.

### FIELD OF THE INVENTION

This invention relates generally to systems and methods for vision testing, and more particularly to systems and methods for measuring aberrations in a patient's vision and in emulating corrective modalities including spectacle lenses to allow the patient to analyze multiple lens designs such as multi-focal spectacle lenses, or progressive add lenses (PALs).

### BACKGROUND

Current vision testing devices that use phoropter technology require that the testing device be positioned intermediate the patient and an image projected on a wall or screen. The phoropter is cumbersome and it commonly introduces instrument accommodation errors in the test results. Moreover, systems that use concave mirrors for reflecting images to the patient typically introduce higher and lower order aberrations since the projected light path and the reflected light path are typically off-axis with respect to an optical axis of the reflective mirror.

Furthermore, systems that measure errors in a patient's vision system and that allow the patient to analyze or compare spectacle lens designs that optimize the patient's vision are nonexistent. For example, there are hundreds of different PAL designs available on the market, and prior art systems provide neither the doctor nor the patient with any practical means to determine, which, if any, design provides the patient with acceptable visual function. Additionally, prior art systems do not allow the patient to preview and compare the visual effects of different PAL lens designs. Nor do prior art systems allow a patient to experience the effects of various lens coatings, such as a photochromic coating, a polarized filter coating, or an antireflective coating.

The present system and methods recognize and address the forgoing considerations, and others, of prior art system and methods.

### SUMMARY OF THE INVENTION

In an embodiment, the invention is directed to systems and methods for measuring a patient's vision and emulating the corrective properties of spectacle lenses. The system comprises one or more or more processors, at least one wavefront modulator operatively coupled to the processor(s) and configured to modulate a wavefront of an image being projected, a patient testing area that has an examination area in which a patient's eyes are to be located when the patient is positioned in the patient testing area, and a reflective mirror having an optical axis that is normal to the face of the reflective mirror where the optical axis is located intermediate the at least one wavefront modulator and the patient examination area. In various embodiments, processor(s) is configured to adjust the at least one wavefront modulator to minimize optical aberrations and errors that result from the optical axis being located intermediate the wavefront modulator and the patient examination area. In various embodiments, the at least one wavefront modulator may be one or more adjustable optical elements that are operatively coupled to, and controlled by, the processor(s).

In another embodiment, a method for correcting off axis errors introduced in an eye examination testing system comprises the steps of projecting a modulated wavefront of an image onto a mirror having an optical axis that is substantially normal to the face of the reflective mirror, reflecting, by the mirror, the modulated wavefront of the image along a reflected light path into an examination area in which the eyes of a patient are located during a vision testing procedure and adjusting, by the at least one processor, the at least one adjustable optical element to minimize one or more optical aberrations and errors introduced by the mirror due to the off-axis incident and reflected light paths. In various embodiments, the incident light path of the modulated wavefront is off-axis with respect to the optical axis, the reflected light path is off-axis with respect to the optical axis, the wavefront of the image is modulated by at least one adjustable optical element, and the at least one adjustable optical element is controlled by at least one processor.

In yet another embodiments, a system for measuring a patient's vision and emulating a corrective lens comprises at least one processor, at least one wavefront modulator operatively coupled to the at least one processor and configured to modulate a wavefront of an image being projected, a patient testing area that comprises an examination area, and a mirror having an optical axis that is normal to the face of the reflective mirror. In various embodiments, the optical axis is located intermediate the at least one wavefront modulator and the patient examination area. In some embodiments, the at least one processor is configured to receive at least one spectacle lens design and adjust the at least one wavefront modulator to modulate at least one image so that the at least one image reflected off the mirror into the patient testing area emulates the corrective characteristics of the at least one spectacle lens design. In some of these embodiments, the at least one processor is configured to receive a plurality of spectacle lens designs, and adjust the at least one wavefront modulator to modulate the at least one image so that the image reflected off the mirror into the patient testing area emulates the corrective characteristics of at least two spectacle lens designs, side-by-side, to allow the patient being tested to preview and compare the at least two spectacle lens designs substantially simultaneously. In some embodiments, the system further comprises a plurality of wavefront modulators and a plurality of images.

### BRIEF DESCRIPTION OF THE DRAWINGS

FIG. 1 is a side view of a vision testing system in accordance with an embodiment of the present system.

FIG. 2 is a perspective view of a patient chair and tower of the vision testing system of FIG. 1.

FIG. 3 is a top view of wavefront modulators for use in the vision testing system of FIG. 1.

FIG. 4 is a detailed view of a wavefront modulator for use in the vision testing system of FIG. 1.

FIG. 5 is a side view of a vision testing system having multiple wavefront modulators in accordance with an embodiment of the present system.

FIG. 6 is a block diagram of inputs and outputs of the system computer.

FIG. 7 shows an image of a patient being tested with the vision system of FIG. 1, with the patient's eyes and direction of gaze being identified by a head, eye and gaze tracking system in accordance with an embodiment of the present system.

FIG. 8 is a perspective view of the vision testing system of FIG. 1 showing a near-viewing accessory in accordance with an embodiment of the present system.

FIG. 9 depicts how a patient can compare both distance and near vision through two different lens designs, B and C, on a simultaneous, side-by-side basis using the vision testing system of FIG. 5.

FIG. 10 is a depiction of three different PAL designs.

FIG. 11 shows three different PAL designs, A, B, and C depicting the power of the lens as a function of vertical gaze angle θ and horizontal gaze angle Δ.

FIG. 12 shows the intersection of the entrance pupil of the eye with the surface of the lens in 15 different positions of gaze A-O for each PAL design A, B, and C.

FIG. 13 is a block diagram showing the method steps carried out by an error correction module of the present system.

### DESCRIPTION OF SOME EMBODIMENTS

Reference will now be made in detail to embodiments of the present systems and methods, one or more examples of which are illustrated in the accompanying drawings. Each example is provided by way of explanation, not limitation of the present system. In fact, it will be apparent to those skilled in the art that modifications and variations can be made to the present systems and methods without departing from the scope or spirit thereof. For instance, features illustrated or described as part of one embodiment may be used in another embodiment to yield a still further embodiment. Thus, the present systems and methods cover such modifications and variations as come within the scope of the appended claims and their equivalents.

### Overview

The present systems and methods are directed generally to a vision testing system that remotely creates and projects a corrected image to the eyes of a patient being tested. In general, the system is comprised of a patient testing unit and a remote located viewport having a reflecting mirror contained therein. The patient testing unit has a patient station, such as an examination chair, and one or more image wavefront modulators located above the patient examination chair in a tower. Each image wavefront modulator contains one or more adjustable optical elements, which in preferred embodiments may be continuously variable power lens (CVPL) elements that modulate the wavefront of an image when the image is projected through the adjustable lens elements. The adjustable CVPL lens elements are based on Alvarez lens pairs that impart spherical corrections, and Humphrey's lens pairs (J90° & J45°) that impart astigmatic corrections to the image wavefront. This embodiment could also include other CVPL elements that correct for higher order axi-symmetrical aberrations. When a projected image is passed through the wavefront modulator, the image wavefront is modulated and directed along an incident light path toward the mirror located in the viewport. In preferred embodiments, the mirror is a spherical concave mirror having an optical axis that is normal to a face of the mirror and a radius of curvature of about 2-2.5 meters.

In preferred embodiments, the distance between the wavefront modulator and the viewport mirror, and the viewport mirror and the patient examination chair are each substantially equal to the radius of curvature of the mirror so that the corrective lenses in the image wavefront generator and the spectacle plane of the patient are optically conjugate approximately the midpoint of the wavefront modulator assembly with respect to the mirror. Moreover, the magnification of the power of the corrective lenses in the image wavefront modulator relative to their emulated power at the spectacle plane under these conditions is 1:1, or unity magnification. In this configuration, optical elements contained in the wavefront modulator are effectively emulated as if the optical elements were located adjacent the patient's eyes. In this way, a patient may have their vision tested without having to place optical elements adjacent their eyes during the testing procedure, thereby permitting vision testing under natural viewing conditions.

Because the wavefront modulator and the patient's eyes are off the optical axis of the viewport mirror, aberrations caused the by mirror's orientation are introduced into the modulated wavefront of the image being viewed by the patient. Thus, in order to minimize the aberrations introduced by the use of the mirror in this off-axis configuration, the system may use calibration data in look-up tables to adjust the optical elements in the image wavefront modulator to correct for these aberrations. Moreover, as a patient moves their head when seated in the examination chair, the distance between the patient's eyes and the viewport mirror may change, causing changes in the effective power of the correcting lenses that are relayed by the mirror. Similar to the means of minimizing off-axis mirror aberrations described above, the system may employ a patient gaze tracking system that can detect and track the position of a patient's eyes. This data may be used by the system computer to determine real-time changes in the distance between the patient's eyes and the viewport mirror. Using this data, the system computer can adjust the optical elements in the wavefront modulator to accommodate for the loss of unity of magnification.

Finally, the viewport mirror may also be mounted using a movable mount that is controlled by the system computer. Thus, as the tracking system detects movement of the patient's head and eyes within the vision testing system, the viewport mirror may be rotated along its vertical and/or horizontal axis to align the reflected light path with the patient's eyes as they naturally move about an examination area.

### Exemplary System Design

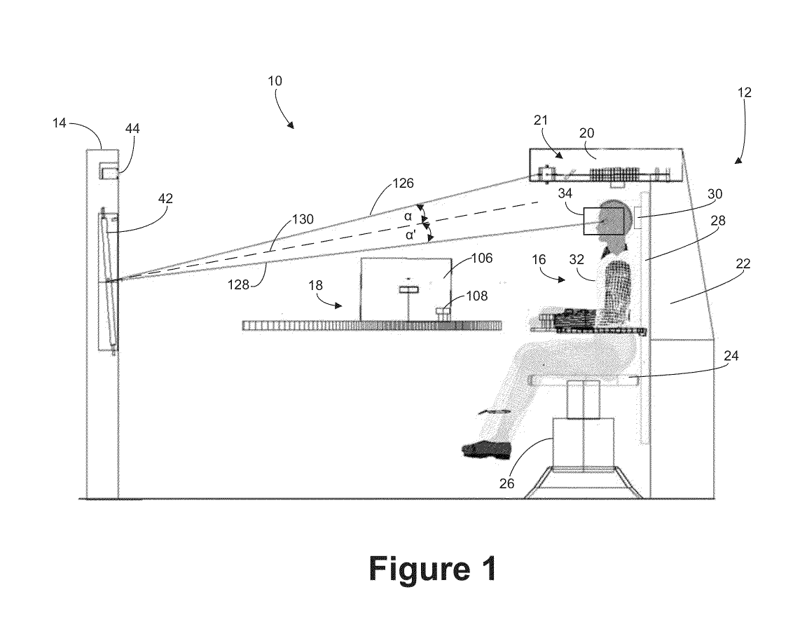

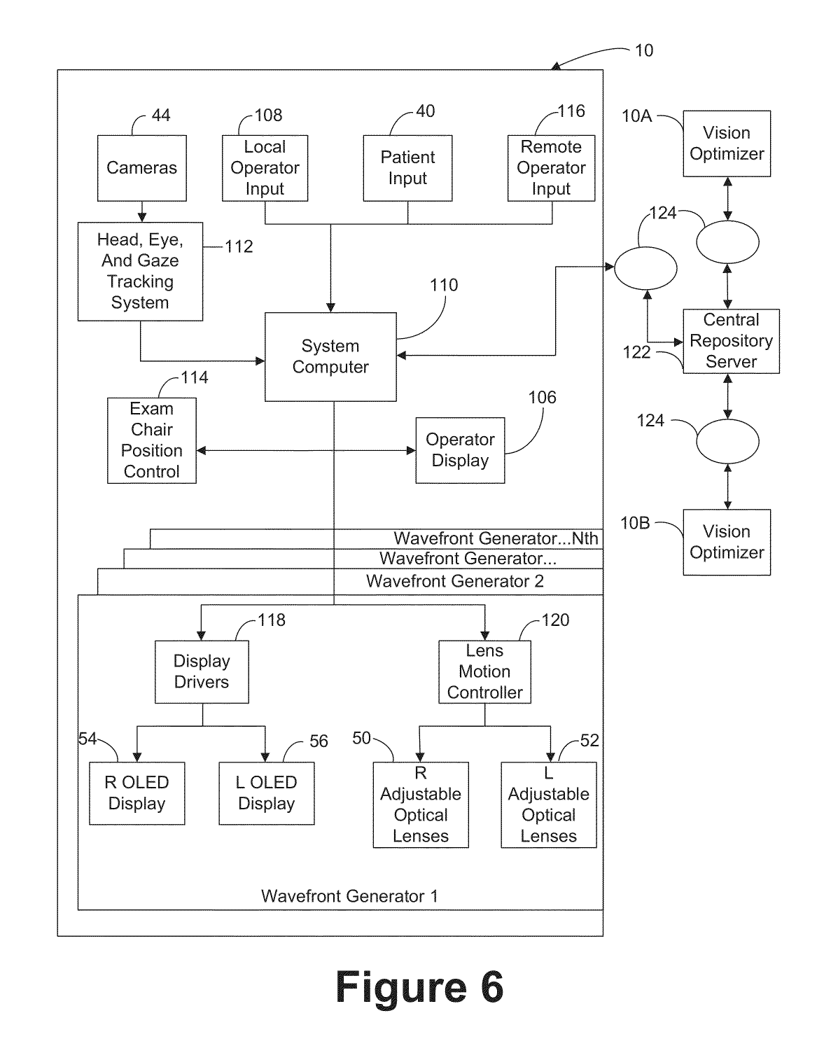

Referring to FIG. 1, a vision testing system 10 is shown having a tower 12, a viewport 14, an examination chair 16, and an operator control terminal 18. Tower 12 has an optical tray 20 that houses one or more wavefront modulators 21. Tower 12 also has a back area 22 that houses a system computer 112 (FIG. 6), a power supply (not shown), and other specialty electronics (not shown) that are operatively coupled to, and that control, the wavefront modulators 21, the examination chair 16, the viewport 14 and the control terminal 18. Separate computers linked in a local network may be used to control any of the above elements.

Examination Chair

The examination chair 16 is located adjacent, and forward of, tower 12 and is preferably mechanically isolated from the tower so that patient movements in the chair are not transmitted to the components in the tower. Examination chair 16 has a seat portion 24, the position of which is adjustable through a motor (not shown) located in a base 26 of examination chair 16. The motor may be adjusted in response to outputs from the system computer. A seat back 28 has a head rest 30 that may be adjustable through manual or by automatic means that is responsive to the system computer. In various embodiments, an optional head restraint (not shown) may be deployed from the underside of optical tray 20 to aid in stabilizing the patient's head during the exam. The examination chair 16 is configured to receive a patient 32 and to position the patient's eyes within an examination area 34.

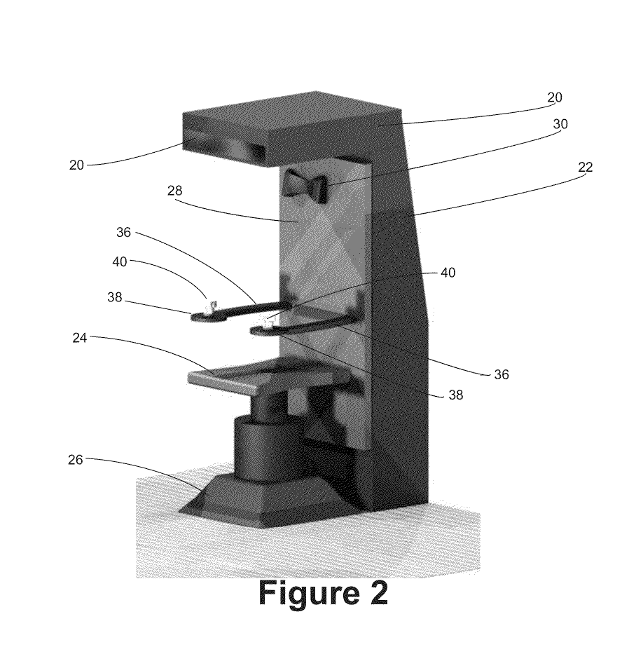

Referring to FIG. 2, examination chair 16 also has arm rests 36, each of which has a platform 38 for supporting a patient input means 40. In a preferred embodiment, input means 40 is a rotary haptic controller that the patient may rotate, translate, or depress to provide input to the system computer during an examination. Suitable haptic controllers are manufactured by Immersion Technologies, San Jose, Calif. 95131, and such controllers are particularly suited to providing intuitive input to the system during an examination. Numerous other input devices are known, such as a mouse, a joystick, a rotary control, touch-sensitive screen or voice controller, any of which may be employed in alternative embodiments.

Wavefront Modulators

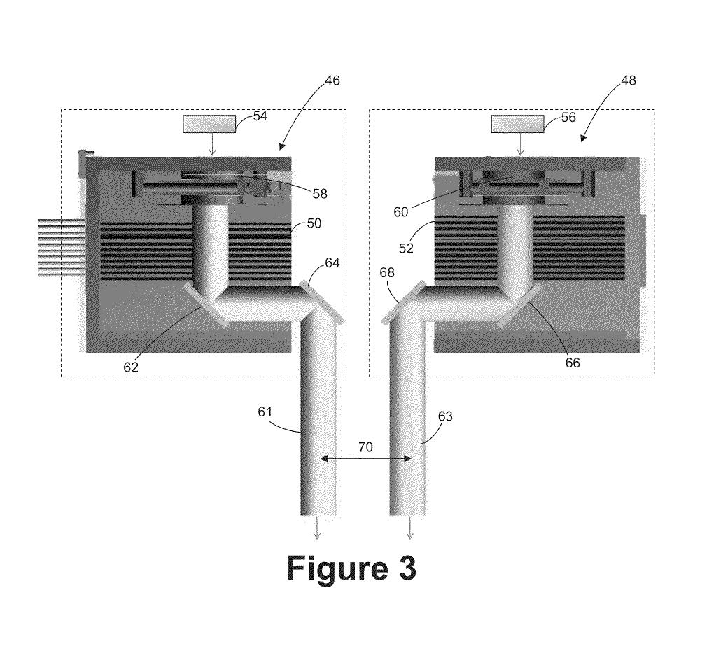

FIG. 3 shows a top view of two particular image wavefront modulators 46 and 48 respectively for a patient's right eye and left eye. Each image wavefront modulator 46 and 48 contains adjustable optical elements and accessory elements 50 and 52 (hereinafter “adjustable optical element”, which may be continuously variable power lens (CVPL) elements). Image generating projectors 54 and 56 (hereinafter “image projectors”) create images that are projected through their respective optical elements, which modulate the wavefront of the image. For the purpose of this invention, the term “images” should be interpreted to mean any static or dynamic image of any color, contrast, shape, or configuration. In various embodiments, image projectors 54 and 56 may be configured to generate images of real-world scenes that are relevant to the patient's lifestyle and these images may be static or full-motion video. One suitable image generating projector is model SXGA OLED-XL™, made by EMagin Company, Bellevue, Wash. Numerous other image generating projectors are known in the art including LED, OLED, DLP, CRT and other light generating technologies, any and all of which may be suitable in alternative embodiments.

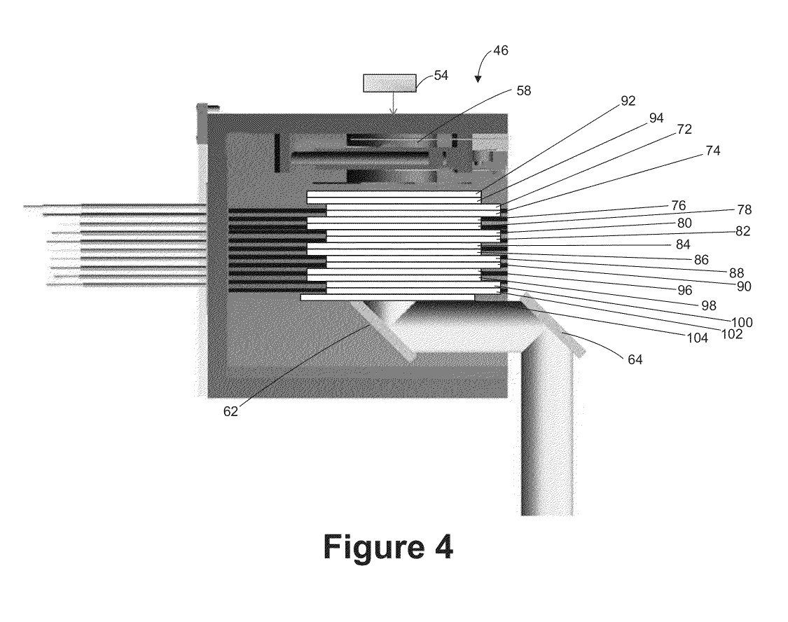

Images generated by projectors 54 and 56 pass through respective collimating lenses 58 and 60 to convert divergent beams of light into parallel light beams. The parallel light beams pass through respective adjustable optical elements 50 and 52 (shown in detail in FIG. 4) to modulate the wavefront of the projected image. Light paths 61 and 63 for the modulated image wavefronts are then redirected by beam turning mirrors 62 and 64 for one eye, and by beam turning mirrors 66 and 68 for the other eye. As the images with modulated wavefronts exit wavefront modulators 46 and 48, light paths 61 and 63 are directed toward field mirror 42 (FIG. 1). In order to properly direct light paths 61 and 63 to field mirror 42 and to adjust the spacing 70 between light paths 61 and 63 to match that of the patient's inter-pupillary distance, the position and angle of lenses 62, 64, 66, and 68 may be adjusted. In various embodiments, lenses 58, 60, 62, 64, 66, and 68 may be coupled to actuators that are responsive to data obtained by tracking system 112 (FIG. 6) to aid in directing light paths 61 and 63 along desired paths for patient testing. In other embodiments, the wavefront modulators 46 and 48 or various optical components therein may be movable to keep the position of adjustable optical elements 50 and 52 at a desired distance from field mirror 42 in order to minimize error due to a loss of unity of magnification as explained below.

Suitable continuous variable power lens (CVPL) elements 50 and 52 for wavefront modulators 46 and 48 include, but are not limited to, Alvarez lenses. In general, each CVPL pair comprises two lens elements, where the surface of each may be described by a cubic polynomial equation and each lens element being a mirror image of its companion lens element. As the lens elements translate relative to each other in a direction that is perpendicular to the optical axis of the elements, the optical power imparted to an image passing through the lens pair changes as a function of the amount of lens translation. Stated differently, Alvarez lens elements modulate the wavefront of the image. Thus, in various embodiments, each lens of the CVPL pair is mounted in a movable frame (not shown) that is operatively coupled to actuators (not shown) that are controlled by system computer 110 (FIG. 6). Examples of actuators that may be used include, but are not limited to, worm screws driven by stepper motors, piezo-electric actuators, and other actuators. One such stepper motor system suitable for the present system is an Arcus NEMA DMX-K-DRV-11-2-1 motor available from Arcus Technologies, Livermore, Calif. 94551. In order to optimize the CVPL elements, the coefficients of the equations that define the shape of the CVPL elements may be optimized to improve their optical performance and to minimize undesirable aberrations of the lens pairs themselves that may result from the lens pairs being aligned in a serial array. Such optimization may be performed, for example, using suitable optical design software such as ZeMax (Radiant ZEMAX LLC, 3001 112th Avenue NE, Suite 202, Bellevue, Wash. 98004-8017 USA).

FIG. 4 shows a detailed view of image wavefront modulator 46 showing adjustable optical elements 50 that are used to modulate the wavefront of the image that is created by image generating projector 54. For purposes of discussion, the embodiment shown in FIG. 4 uses continuously variable power lenses—Alvarez lenses. In particular, a first lens pair 72 and 74 may be elements that provide correction for spherical power—Alvarez lenses. A second lens pair 76 and 78 may be 0°-90° Jackson cross cylinder elements—Humphrey's lenses. A third lens pair 80 and 82 may be 45°-135° Jackson cross cylinder elements—Humphrey's lenses. The cross cylinder elements provide correction for cylindrical power. A fourth lens pair 84 and 86 may be for spherical aberration. Finally, a fifth lens pair 88 and 90 may be for comatic aberration. The remaining lenses 92-104 may be accessory lenses such as a polarized lens and various other lenses having lens coatings (e.g., photochromic coatings, antiglare coatings, etc.). Each of the lens pairs modulates the wavefront of an image as the image is projected through wavefront modulator 46. Each of the accessory lenses with a particular coating further modifies the image according to the properties of the coating. Adjustable optical elements 72-90 may be selected to provide a full range of correction of refractive errors from −20D to +20D and astigmatic corrections up to, or beyond, 8D. As a result, in addition to adjustable optical elements 50 providing corrections for spherical and cylindrical power, the adjustable optical elements may also be able to correct for higher order aberrations of a range that is suitable to the application of the instrument.

In addition to including accessory lenses in adjustable optical elements 50, phase plates, such as those prepared by lathing the surface of a PMMA disc or other suitable optical material into the desired shape, may also be inserted in accessory slots 92-104. These phase plates may be used to impart additional modulation to the wavefront of the image that may be necessary to emulate the spectacle lens design being emulated. Furthermore, adjustable optical elements 50 may also be used to emulate the optical properties of contact lenses, intraocular lenses, as well as various refractive surgery profiles, such as LASIK or PRK, to allow a patient to evaluate the effectiveness of each potential vision correcting option presented to the patient.

It should be understood from reference to this disclosure that other types of adjustable optical elements and mirrors may be used in wavefront modulators 46 and 48. For example, wavefront modulators 46 and 48 may use fixed and adjustable lens elements to modulate spherical and astigmatic errors, and deformable mirror elements to impart higher order aberrations to the wavefront of the image. Such deformable mirrors that may be responsive to a computer are manufactured by Edmunds Optics, 101 East Gloucester Pike, Barrington, N.J. 08007-1380. In still other embodiments, the adjustable CVPL described above may be replaced by fixed lenses, by one or more deformable mirrors, or by any combination of fixed lenses, deformable mirrors, and CVPL elements. In various embodiments, adjustable CVPL elements may be employed to correct for lower order aberrations of spherical error and astigmatism, and deformable mirrors may be employed to correct for higher order aberrations thereby using the dynamic range of the adjustable mirrors only for creating higher order corrections.

Viewport

Referring once again to FIG. 1, viewport 14 houses a reflective field mirror 42 and one or more patient tracking cameras 44. In various embodiments, tracking cameras 44 are operatively coupled to a head, eye, and gaze tracking system 112 (FIG. 6) that uses information provided by tracking cameras 44 to measure features of the patient (e.g., pupillary distance, eye position, patient position, etc.). In various embodiments, field mirror 42 is round in shape and has a spherical concave curvature with a radius of curvature of approximately 2.5M and a diameter of between 10″ to 24″. A suitable mirror may be procured from Star Instruments, Newnan, Ga. 30263-7424. In other embodiments, the system may include the use of an aspheric mirror, a toroidal mirror, a mirror that is non-circular in shape, or a plano mirror.

In embodiments that use a concave spherical field mirror 42, a distance between a spectacle plane adjacent the patient's eyes (at examination area 34) to field mirror 42 and from the center of adjustable optical elements 50 and 52 to field mirror 42 should each be approximately equal to the radius of curvature of the mirror. In this configuration, the corrective lenses in the image wavefront modulator and the spectacle plane are optically conjugate with respect to the field mirror. Moreover, the magnification of the image relative to the object under these conditions is 1:1 or unity magnification. Because wavefront modulators 46 and 48 and the examination area 34 are located at optical planes that are substantially conjugate with respect to the field mirror, adjustable optical elements 50 and 52 are optically relayed to the spectacle plane located in examination area 34 and produce the same effective power at spectacle plane as they produce in the wavefront modulators. Thus, a patient seated in vision testing system 10 views the image as if adjustable optical elements 50 and 52 are positioned adjacent their eyes.

Vision Testing System with Compare Features

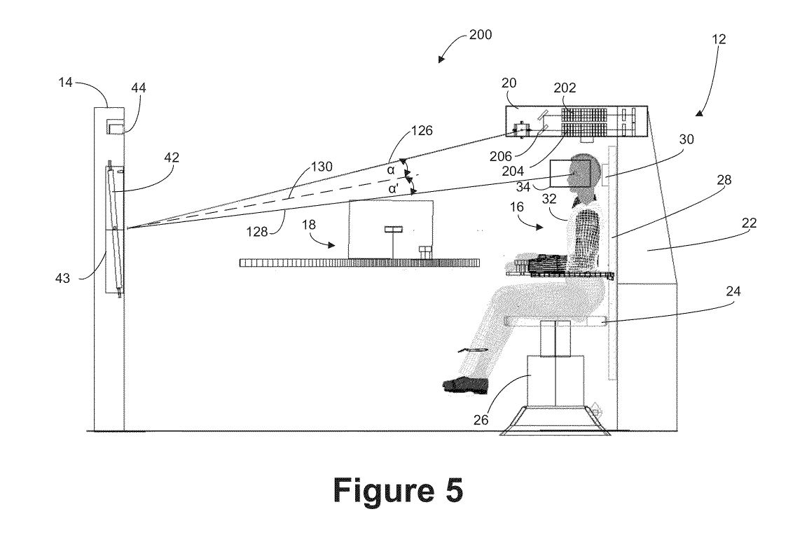

FIG. 5 shows a side view of another embodiment of a vision testing system 200 in which two wavefront generators 202 and 204 per eye, four in total, are housed in optical tray 20. Thus, modulated wavefronts of images from upper wavefront modulator 202 and lower wavefront modulator 204 are combined by beam combining element 206 and thereafter directed along an incident light path 126 out the wavefront modulators towards field mirror 42. Similar to that described with respect to FIG. 1, the modulated image wavefronts are reflected off of field mirror 42 along a reflected light path 128 into examination area 34. As will be described below, a plurality of wavefront generators per eye not only allows the patient to compare potential corrections, but it also allows the patients to view and compare images that would be produced by a plurality of spectacle lens designs on a side-by-side and simultaneous, or substantially simultaneous, basis permitting the patient to select the image that is deemed to be of the best quality, or otherwise preferred.

Control Terminal

Referring once more to FIG. 1, operator control terminal 18 may comprise a touch display terminal 106 that is used by the operator to provide control inputs to system computer 110 (FIG. 6) and to receive displays from the system computer. The system may also receive inputs from the operator by a conventional input device 108 (e.g., a keyboard, mouse, or haptic dial) to control the vision testing system during the examination. Touch display 106 and input device 108 are connected to system computer 110 (FIG. 6) through conventional cable, fiber optic, or wireless connections.

FIG. 6 shows a schematic diagram of vision testing system 10 that includes system computer 110 operatively coupled to various subsystems. For purposes of this disclosure, reference to system computer 110 should be understood to include one or more system computers that are operatively connected and configured to carry out the described functionality. In particular, system computer 50 receives patient tracking information from tracking system 112, which uses information received from tracking cameras 44 to determine three-dimensional head, eye and gaze information. The head, eye and gaze information may be used by system computer 110 to adjust the adjustable optical elements 50 and 52 to correct for errors introduced by movement of the patient's head within examination area 34.

System computer 110 is also configured to receive inputs from touch display 106 and operator input device 108. These inputs may be used to control the position of examination chair 16 by way of exam chair position control unit 114 to ensure that the patient's eyes are properly positioned in the examination area 34. In some embodiments, operator input may be received via remote control inputs such over an Internet connection 116 when the operator is located remote to vision testing system 10. Moreover, system computer 110 is also configured to receive patient input from patient input means 40. In this way, the patient can provide various inputs during an examination that would cause system computer 110 to adjust respective adjustable optical elements 50 and 52. In this way, the system may be configured to use patient input to facilitate the examination.

In addition to receiving inputs from various subsystems (e.g., the patient and operator controls and the tracking system), system computer 110 also provides outputs to a display driver 118 that drives image projectors 54 and 56. System computer 110 also provides outputs to a lens motion control system 120 that directs the actuators (not shown) that drive the respective adjustable optical lenses 50 and 52 for the right and left channels of the wavefront modulators 46 and 48, respectively. Lens motion controller 120 also controls the position of accessory lenses 92-104.

In addition to receiving local inputs and sending local outputs, system computer 110 may also be operatively coupled to a central repository server 122 over a network connection 124 (e.g. the Internet, wide area network or cellular network). Moreover, in some embodiments, multiple vision testing systems 10A and 10B may be operatively coupled to central repository server 122 over networks 124. Server 122 may comprise an information storage device, such as, for example, a high-capacity hard drive or other non-volatile memory devices to allow patient data to be stored and transmitted to lens manufacturing facilities. Server 122 may also be configured to respond to queries from one or more of the vision testing systems 10, 10A and 10B and may provide any requested service such as performing statistical analysis on data obtained by the vision testing systems.

### Exemplary System Operation

Referring once again to FIG. 1, patient 32 occupies examination chair 16, which is positioned below optical tray 20. The operator, using touch display 106 or input means 108, adjusts the position of seat 24 to move the patient's eyes within examination area 34. Images generated by projectors 54 and 56 are passed through image wavefront modulators 46 and 48 in optical tray 20, where the image wavefront is modulated by adjustable optical elements 50 and 52. The images are then directed along the incident light path 126 toward viewport 14. The modulated image wavefront is reflected off of field mirror 42 along a reflected light path 128 toward examination area 34 where the patient's eyes are located. In the configuration shown in FIG. 1, the incident light path 126 is offset from an optical axis 130 of field mirror 42 by an angle α. Moreover, the reflected light path 128 is also offset from optical axis 130 by substantially the same angle α′. It should be understood by reference to this disclosure that the angle α′ may change slightly as the patient moves their head within examination area 34. Furthermore, if the patient's eyes are not in the same plane as wavefront modulators 46 and 48, a second angle β (not shown) that is perpendicular to the angles α and α′ is also present. The second angle β occurs when the patient moves their head left to right off of optical axis 130 when seated in examination chair 16.

Astigmatism, higher order aberrations and other optical errors may be introduced into vision testing system 10 in various ways. For example, off axis angles α, α′ and β induce astigmatism and higher and lower order aberrations into the modulated image wavefronts. In various embodiments, these aberrations may be compensated for, completely, or in part, by adjusting the appropriate adjustable optical elements 50 and 52 in respective wavefront modulators 46 and 48. That is, one or more of the lens pairs 76-90 can be adjusted to eliminate or minimize the aberrations that are introduced by off-axis incident and reflected light paths. Moreover, because α, α′ and β may change as the position of the patient's eyes move about examination area 34, system computer 110 (FIG. 6) may use information provided by tracking system 112 to dynamically change adjustable optical elements 50 and 52 to compensate for the aberrations that occur due to the patient's head movement. Such adjustments ensure that the measurement of refractive errors, aberrations, and the emulation of corrections remain accurate as the position of the patient's eyes move about examination area 34.

As previously indicated, operating vision testing system 10 at, or near, the condition of unity magnification is preferred. However, unity magnification is not always possible since the patient is free to move about examination area 34 during testing. That is, as the patient's eyes move toward and away from field mirror 42, changes in the effective lens power may result. Vision testing system 10 may compensate for such changes in effective lens power through use of the following equation:

Po=Pc(M)²

where Po is the effective power of the lens at the patient's spectacle plane, Pc is the actual power of the corrective lenses, and M is the magnification, given by Di/Do, where Do is the distance between the corrective lenses and the field mirror and Di is the distance between the field mirror and the patient's eyes. The above formula provides corrective conversions that may be stored in calibration tables and used by system computer 110 to adjust one or more lenses in adjustable optical elements 50 and 52 to correct for such non-unity magnifications. Such corrections may be automatically made by system computer 110 without input by the operator by using patient tracking information data provided by tracking cameras 44 and tracking system 112.

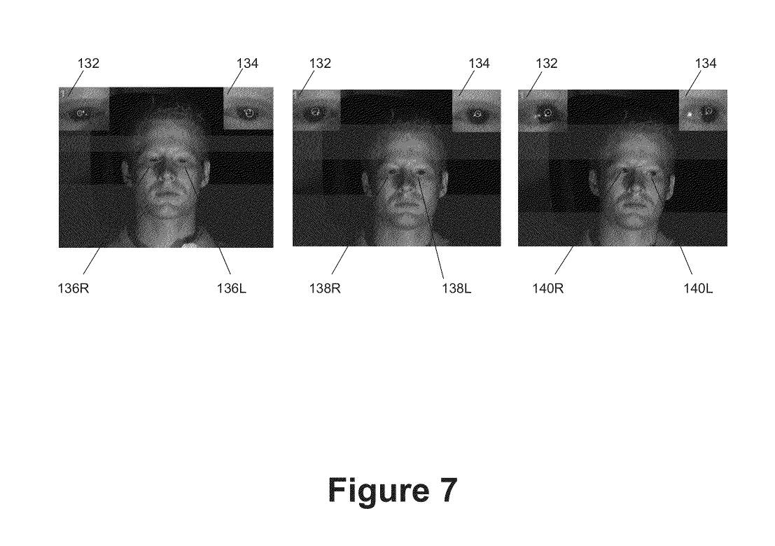

Referring to FIG. 7, tracking system 112 captures an image of the patient's head using tracking cameras 44 and identifies the positions of the patient's right eye 132 and left eye 134. In a preferred embodiment, tracking cameras 44 are sensitive to infrared (IR) light and IR illuminators are located to the patient's right and left (not shown). The IR illuminators are configured to direct IR light into the patient's eyes so that IR light reflected by the patient's corneas can be detected by tracking cameras 44. Thus, reflection of images produced by the IR illuminators, of known geometry and position, are used by tracking system 112 to measure the distance between patient 32 and field mirror 42. In various embodiments, two or more tracking cameras 44 may be located some distance apart, providing stereo-scopic measurement capabilities to improve distance measurements. By comparing the size and location of the patient's pupil and the size and location of the IR images reflected by the cornea, it is possible for tracking system 112 to compute a direction of gaze by taking the center of the corneal spheroid and the center of the pupil and computing a vector that connects these two points in space, which provides the system with an accurate direction of patient gaze. Examples of gaze direction vectors for each eye, computed separately and in different fields of gaze, are shown as 136R. 136L. 138R, 138L, 140R and 140L. Tracking system 112 may compute off axis angles θ (vertical) and Δ (horizontal) for each position of gaze. These angles are a function of both the position of the patient's head and the position of the eyes.

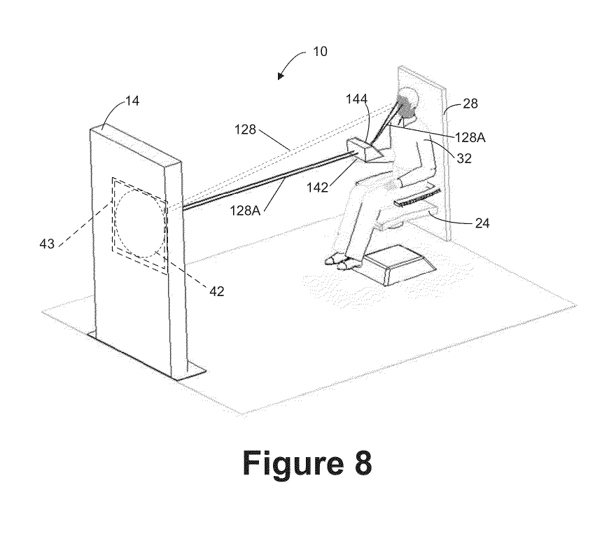

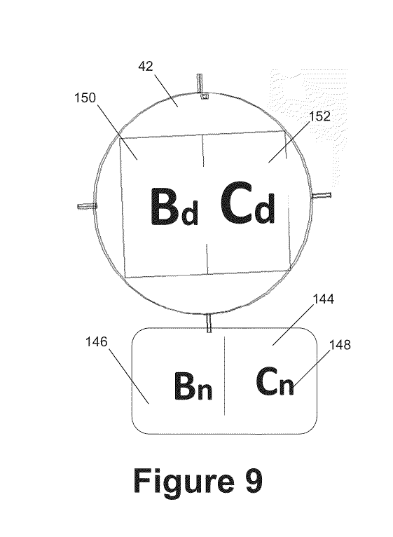

Referring to FIGS. 8 and 9, vision system 10 is shown in use with the wavefront modulators removed for clarity with a near-viewing display apparatus 142, which allows a patient to view an image in their near field. That is, reflected light path 128 may be diverted by moving field mirror 42 using a movable mounting 43 that allows the field mirror to rotate about its horizontal and vertical axes. Thus, when near-viewing apparatus 142 is in use, field mirror 42 rotates about its horizontal axis so that a reflected light path 128A is diverted into the back of near-viewing apparatus 142, which redirects the reflected modulated image wavefront to the patient's eyes via a viewing surface 144. That is, mirrors (not shown) inside near-viewing apparatus 142 redirect the reflected light path 128A to the patient's eye. The mirrors (not shown) inside near-viewing apparatus 142 cause the modulated images to diverge with respect to each other, and to appear to the patient in the exam chair as if they emerged from viewing surface 144 of the near-viewing apparatus 142. In this way, the near-viewing apparatus 142 emulates a near field image to allow a patient to experience the vision corrections provided by bi-focal or PAL lenses.

FIG. 9 shows the patient's right eye view of field mirror 42 and viewing surface 144 of near-viewing apparatus 142. In embodiments having two or more wavefront generators per eye (FIG. 5), the patient is able to preview and compare images produced by spectacle lens design B and C simultaneously, on a side-by-side basis, at a close distance through near-viewing apparatus 142, images Bn 146 and Cn 148, and at a far away viewing distance through field mirror 42, images Bd 150 and Cd 152. As such, a patient can simultaneously evaluate lens designs that provide for nearby and far away viewing.

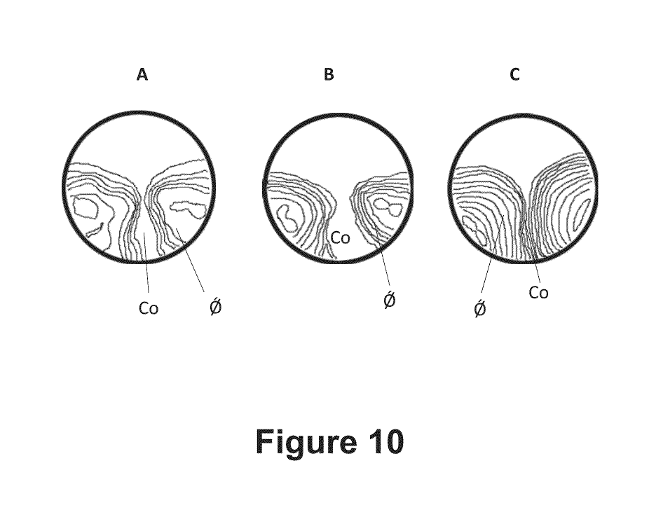

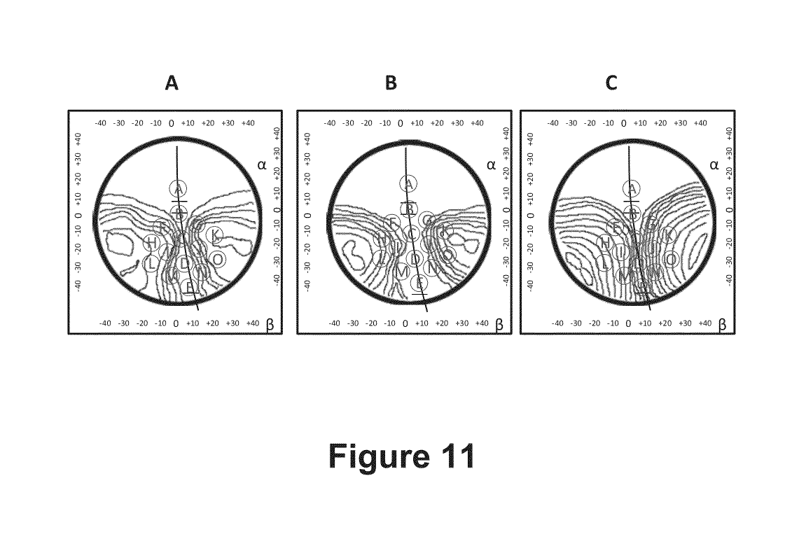

FIG. 10 shows a plan view of three different multi-focal lens designs A, B, and C. The lines \acute{Ø} connect regions of similar optical power. Typical progressive lenses have increasing add power down a central channel of the lens that is known as the corridor Co and increasing levels of astigmatism are found in the lower corners of the lens. Power labels are omitted from FIG. 10 for clarity. As stated previously, tracking system 112 can be used to compute angles θ (horizontal) and Δ (vertical) for each position of patient gaze. Gaze angles θ (horizontal) and Δ (horizontal) are a function of both the position of the patient's head and eyes. As such, the portion of a surface of a spectacle lens intersected by the patient's gaze angles are shown for each PAL lens design in FIG. 11, with the cardinal gaze vector when looking at infinity designated as angle (0, 0) as a function of gaze angles θ and Δ. Instead of angles, the position on the spectacle lenses may also be shown in millimeters (mm) of distance from the optical center of the lens. With a vertex distance of approximately 14 mm, 20 degrees of gaze angle equates to about 1 mm of transverse distance on the spectacle lens.

Vision testing system 10 may be configured to simulate a progressive lens by modulating the image wavefront based on the lens design. For example, a progressive lens design that describes a unique value of sph, cyl, and HOA for a region of the lens that is subtended by the eye's entrance pupil for each gaze angle pairs θ and Δ may be loaded into system computer 110. The lens design may be provided by a lens manufacturer, measured by an appropriate lens mapper, or measured by a spatially resolved refractometer, which may be provided as an accessory to vision testing system 10. The lens information may then be used to modulate the wavefront of the image in order to simulate the properties of the lens design for the patient as a function of the gaze angles.

In various embodiments, as the patient's gaze angles change, system computer 110 uses information received by tracking system 112 to compute the gaze angle pair at a rate of, for example, 10-30 Hz, and uses the tracking information to drive lens motion controller 120 to adjust adjustable optical elements 50 and 52 in respective wavefront modulators 46 and 48 to accurately replicate the power of the PAL design exactly as if the patient were wearing the progressive lens and was looking through it at the measured gaze angle. Examples of the area of the lens surface subtended by different gaze angles is shown in FIG. 11, with the different lens positions subtended indicated by letters A-M, for each lens design A, B, C. Because tracking system 112 and lens motion controller 120 work at rapid rates, vision testing system 10 provides the patient with realistic simulation of a progressive lens design as the patient's gaze angle changes with natural head and eye movements.

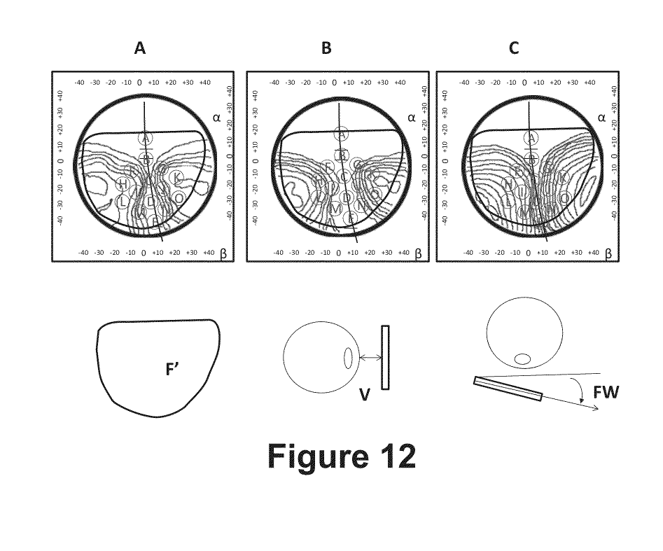

As shown in FIG. 12, by loading the patient's fitting information from a selected frame F′ into system computer 110 in addition to the spectacle lens design, including the vertex distance V and the frame wrap angle FW, vision system 10 may further enhance the accuracy of the spectacle lens simulation as viewed by the patient. That is, the values of V and FW influence the effective optical power and aberrations for each surface point of the lens subtended by the entrance pupil.

### Exemplary Error Correction Module Operation

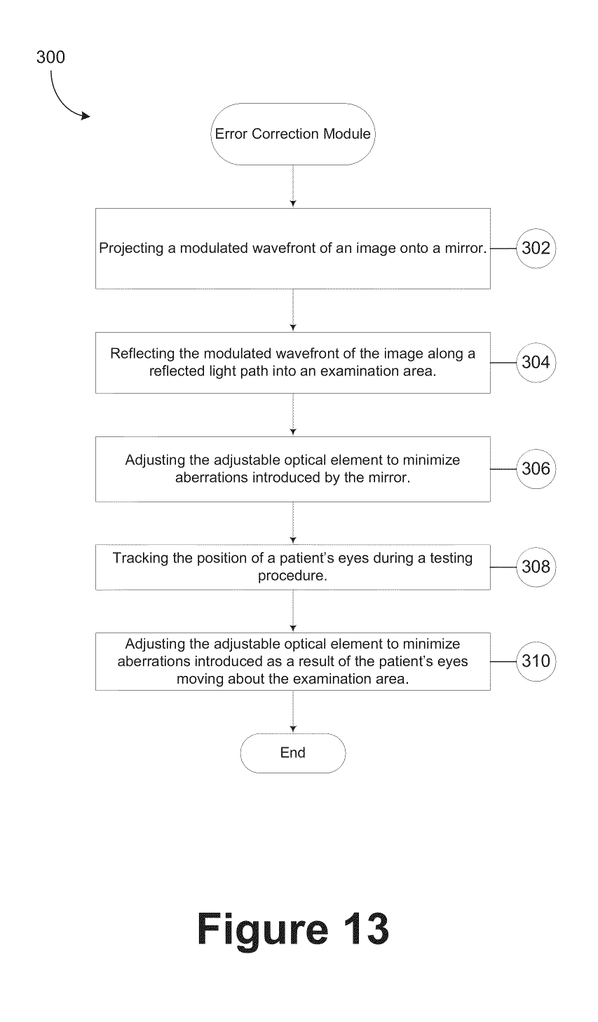

FIG. 13 depicts exemplary methods for correcting higher and lower order aberrations that are introduced by: (1) incident 126 and reflected 128 light paths that are off-axis with respect to the optical axis 130 of field mirror 42 and effective power changes due to movement of the patient during testing. It should be understood by reference to this disclosure that the error correction module 300 describes exemplary embodiments of the method steps carried out by the present system, and that other exemplary embodiments may be created by adding additional steps or by removing one or more of the methods steps described in FIG. 3.

At step 302, image projectors 54, 56 (FIG. 3) project an image through a corresponding wavefront modulator 46, 48, which directs the modulated image wavefront toward mirror 42 (FIG. 1) having optical axis 130 that is normal to the face of the mirror. An incident light path 126 of the modulated image wavefront is off-axis with respect to the optical axis 130 of the field mirror. The wavefront modulator may have one or more adjustable optical elements 50, 52 (FIG. 3) that are controlled by system computer 110 (FIG. 7).

At step 304, the modulated wavefront of the image is reflected by mirror 42 along a reflected light path 128 that is also off-axis with respect to optical axis 130. In various embodiments, mirror 42 may be a concave spherical mirror, which imparts various higher order and lower order aberrations into the modulated wavefront of the image when the incident and reflected light paths are off-axis with respect to the mirror's optical axis. Thus, at step 306, the system computer 110 may be configured to adjust optical elements 50, 52 in respective wavefront modulators 46, 48 to minimize aberrations introduced by the mirror. The adjustment factors may be determined during calibration of vision testing system 10 and stored in calibration look-up tables.

In various embodiments, at step 308, the system is configured to track the position of a patient's, head, eyes and gaze using tracking system 112. The position of the patient's head, eyes and gaze may be used to determine the locations of the patient's eyes with respect to wavefront modulator 46, 48, mirror 42 and reflected light path 128. In various embodiments, at step 310, system computer 110 may be configured to use the data calculated by tracking system 112 to adjust optical elements 50, 52 to minimize aberrations and errors (e.g., changes in the effective lens power) introduced as a result of the patient's eyes moving out of the conjugate plane with optical elements 50, 52, thereby resulting in a loss of unity magnification between the adjustable lenses and the present location of the patient's spectacle plane. Once more, system computer 110 may use calibration data stored in look-up tables to impart the appropriate adjustments to optical elements 50, 52 to accommodate for patient movement within the vision testing device.

In various embodiments, movable mirror mounting 43 coupled to field mirror 42 and to system computer 110 may be used to align reflected light path 128 with the patient's eyes as the patient move about examination area 34. In this way, as eye tracking data is obtained by tracking system 112, system computer 110 may cause the movable mirror mount to pivot mirror 42 about its vertical and horizontal axis in an effort to move reflected light path 128 (FIG. 1) in conjunction with movement of the patient's eyes. In this way, the angle of incidence and the angle of reflection of the light path may be maintained with respect to the patient to minimize aberrations introduced by the optical system and mirror.

### CONCLUSION

The present systems and methods provide for a vision testing system that measures optical errors (e.g., lower order and higher order aberrations) in a patient's vision system without having to dispose optical lenses or instruments adjacent the patient's face. Moreover, the system allows a patient to preview and compare potential optical corrections and to select an optimum solution. Moreover, the system may also allow the patient to compare multiple lens designs to determine which design provides the best quality of image or that is otherwise preferred. These images may be compared simultaneously or substantially simultaneously on a side-by-side basis. Thus a plurality of spectacle lenses may be emulated simultaneously or perceived simultaneously by the patient. By activating a wavefront modulator for each eye, a binocular comparison of images for each lens can be previewed and compared for each spectacle lens design. As a result, systems and methods are provided to characterize the optical properties of any spectacle lens, and to accurately emulate those optical properties for a patient under realistic viewing conditions over near, intermediate, and far away distances and over a range of image illuminations, colors and contrasts. By adjusting the output of the image projectors, patients can see how the spectacle lens designs compare as illumination and contrast rises or fall and as colors change. This allows the patient to preview, compare, and select a particular spectacle lens design or feature that they prefer based upon the patient's subjective appraisal.

By using a head, eye and gaze tracking system, the system can stabilize the image into the appropriate image plane, thereby relieving the patient of the need to hold still during the test and facilitates a more realistic emulation of spectacle lens performance under natural viewing conditions. The testing is also done with no instruments or other visual obstructions in the patient's field of view. Optical parameters used to manufacture or select spectacle lenses can be determined in much higher resolution increments, such as 0.01D, as opposed to the 0.25D increments provided by prior art systems and methods.

Many modifications and other embodiments of the disclosed system and method will come to mind to one skilled in the art having the benefit of the teachings presented in the foregoing descriptions and the associated drawings. While examples discussed above cover the use of the invention in the context of a vision testing system, the invention may be used in any other suitable context such as emulating vision correction by spectacle lenses, contact lenses, intraocular implants and Lasik surgery. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for the purposes of limitation.

What is claimed:

1. A system for measuring a patient's vision comprising:
a. at least one processor;
b. at least one image wavefront modulator operatively coupled to the at least one processor and configured to modulate a wavefront of an image being projected;
c. a patient testing area that comprises an examination area, wherein the examination area comprises the area in which a patient's eyes are to be located when the patient is positioned in the patient testing area; and
d. a reflective mirror having an optical axis that is normal to the face of the reflective mirror, wherein the optical axis is located intermediate the at least one wavefront modulator and the patient examination area;
wherein the at least one processor is configured to adjust the at least one wavefront modulator to minimize optical aberrations and errors that result from the optical axis being located intermediate the wavefront modulator and the patient examination area.

2. The system of claim 1, wherein the at least one wavefront modulator further comprises adjustable optical elements selected from a group consisting of:
a. continuously variable power lenses;
b. deformable mirrors;
c. one or more discrete lenses;
d. phase plates;
e. the combination of one or more of a, b. c, or d.

3. The system of claim 1, wherein the optical aberrations and errors are one or more optical aberrations and errors selected from a group consisting of:
a. spherical defocus;
b. astigmatism aberrations; and
c. higher order aberrations.

4. The system of claim 1, wherein the patient testing area further comprises a seat that is operatively coupled to the at least one processor and is configured to be moved to properly locate a patient's eyes in the examination area.

5. The system of claim 1, further comprising a tracking system operatively coupled to the at least one processor, wherein the tracking system is configured to track the eyes of a patient being tested as the patient's eyes move about the examination area.

6. The system of claim 5, wherein the at least one processor is configured to dynamically adjust the at least one wavefront modulator based on data received from the tracking system to minimize the optical aberrations and errors that are introduced by the reflective mirror and from a loss of unity magnification as the eyes of a patient being tested move about the examination area.

7. The system of claim 5, further comprising a movable mounting that is:
a. adapted to couple to the reflective mirror; and
b. operatively coupled to the at least one processor,
wherein the movable mounting moves the reflective mirror based on eye location data obtained by the tracking system.

8. The system of claim 1, wherein the at least one processor is configured to adjust the at least one wavefront modulator so as to emulate the corrective characteristics of at least one spectacle lens design on an image passing through the at least one wavefront modulator.

9. A system for measuring vision comprising:
a. at least one processor;
b. a reflective mirror having an optical axis that is normal to the face of the reflective mirror;
c. adjustable optical elements that are operatively coupled to the at least one processor and configured to modulate a wavefront of an image being projected through the adjustable optical elements onto the reflective mirror, wherein an incident light path between the modulated wavefront and the reflective mirror is off-axis with respect to the optical axis of the reflective mirror; and
d. a reflected light path from the reflective mirror that is off-axis with respect to the optical axis of the reflective mirror;
wherein the at least one processor is configured to adjust the adjustable optical elements to minimize optical aberrations and errors that are introduced to the modulated wavefront due to the off-axis angle of the incident and reflected light paths.

10. The system of claim 9, wherein the reflective mirror further comprises a spherical concave curvature.

11. The system of claim 9, wherein the errors and aberrations are one or more errors and aberrations selected from a group consisting of:
a. spherical defocus error;
b. cylindrical error; and
c. higher order aberrations.

12. The system of claim 9, wherein the reflected light path is substantially located in an examination area where a patient's eyes are to be positioned during vision testing.

13. The system of claim 12, further comprising a tracking system that is operatively coupled to the at least one processor and that is configured to detect and track the eyes of a patient when a patient is being tested.

14. The system of claim 13, wherein the adjustable optical elements are adapted to dynamically minimize one or more of optical errors and aberrations caused by movement of a patient's eyes about the examination area when the patient is being tested.

15. The system of claim 13, further comprising a movable mounting that is coupled to the reflective mirror, wherein the movable mounting is operatively coupled to the at least one processor and configured to move the reflective mirror based on eye tracking data obtained by the tracking system.

16. The system of claim 9, wherein the at least one processor is configured to adjust the adjustable optical elements so as to emulate the corrective characteristics of at least two spectacle lens designs on an image passing through the adjustable optical elements to allow a patient being tested to preview and compare the at least two spectacle lens designs.

17. A method for correcting off axis errors introduced in an eye examination testing system comprising:
a. projecting a modulated wavefront of an image onto a mirror having an optical axis that is substantially normal to the face of the reflective mirror, wherein
i. the incident light path of the modulated wavefront is off-axis with respect to the optical axis,
ii. the wavefront of the image is modulated by at least one adjustable optical element, and
iii. the at least one adjustable optical element is controlled by at least one processor;
b. reflecting, by the mirror, the modulated wavefront of the image along a reflected light path into an examination area in which the eyes of a patient are located during a vision testing procedure, wherein the reflected light path is off-axis with respect to the optical axis; and
c. adjusting, by the at least one processor, the at least one adjustable optical element to minimize one or more optical aberrations and errors introduced by the mirror due to the off-axis incident and reflected light paths.

18. The computer-implemented method of claim 17, wherein the at least one adjustable optical element comprises a plurality of movable Alvarez lenses.

19. The computer-implemented method of claim 17, further comprising
a. tracking, by a tracking system, the position of the patients eyes; and
b. adjusting, by the at least one processor, the at least one adjustable optical element to minimize one or more optical aberrations and errors introduced as a result of the patient's eyes moving about the examination area.

20. The computer-implemented method of claim 19, wherein the step of adjusting the at least one adjustable optical element further comprises automatically adjusting the at least one adjustable optical element in response to the patient's eyes moving about the examination area.

21. The computer-implemented method of claim 17, further comprising:
a. tracking, by a tracking system, the position of the patients eyes; and
b. moving the mirror based on tracking data obtained by the tracking system so as to maintain alignment of the reflected light path with the patient's eyes.

22. The computer-implemented method of claim 21, further comprising adjusting, by the at least one processor, the at least one adjustable optical element to minimize one or more optical aberrations and errors introduced by movement of the patients eyes about the examination area.

23. The computer-implemented method of claim 17, further comprising
a. receiving, by the at least one processor, at least one spectacle lens design; and
b. adjusting the at least one adjustable optical element based on the received at least one spectacle lens design to emulate the corrective characteristics provided by the at least one spectacle lens design.

24. A system for measuring a patient's vision and emulating a corrective lens comprising:
a. at least one processor;
b. at least one wavefront modulator operatively coupled to the at least one processor and configured to modulate a wavefront of an image being projected;
c. a patient testing area that comprises an examination area; and
d. a mirror having an optical axis that is normal to the face of the reflective mirror, wherein the optical axis is located intermediate the at least one wavefront modulator and the patient examination area;
wherein the at least one processor is configured to:
i. receive at least one spectacle lens design;
ii. adjust the at least one wavefront modulator to modulate at least one image so that the at least one image reflected off the mirror into the patient testing area emulates the corrective characteristics of the at least one spectacle lens design.

25. The system of claim 24, wherein the at least one processor is further configured to:
a. receive a plurality of spectacle lens designs; and
b. adjust the at least one wavefront modulator to modulate the at least one image so that the image reflected off the mirror into the patient testing area emulates the corrective characteristics of at least two spectacle lens designs side-by-side to allow the patient being tested to preview and compare the at least two spectacle lens designs substantially simultaneously.

26. The system of claim 25, further comprising a plurality of wavefront modulators and a plurality of images.
